(19) United States
(12) Patent Application Publication
(10) Pub. No.: US 2010/0082051 A1
(43) Pub. Date: Apr. 1, 2010

(54) VASCULAR OCCLUSION DRILL

(76) Inventors: PATRICIA E. THORPE, IOWA CITY, IA (US); FRANCISCO J. OSSE, SAO PAOLO (BR); CHARLIE CAVE, PRESCOTT, AZ (US)

Correspondence Address: BULLIVANT HOUSER BAILEY PC, 1415 L STREET, SUITE 1000, SACRAMENTO, CA, 95814, US

(21) Appl. No.: 12/563,968

(22) Filed: Sep. 21, 2009

Related U.S. Application Data
(60) Provisional application No. 61/098,684, filed on Sep. 19, 2008.

(51) Int. Cl. A61B 17/22

(52) U.S. Cl. 606/159

(57) ABSTRACT

The invention provides a medical device for treatment of an obstructed vessel, the apparatus comprising: (a) an external conduit comprising a proximal end, a distal end, a cylindrical outer surface sized for passage through a blood vessel, and an internal lumen, the conduit lumen being threaded within at least a portion of the length of the conduit at the distal end, and (b) a shaft comprising a proximal end, a distal end, an internal lumen, and a cylindrical outer surface fitted inside the conduit lumen and having external threading over at least a portion of the length of the shaft at the distal end; where the shaft threading engages the external conduit threading allowing the shaft to be advanced through the lumen by rotation of the shaft.

### PRIORITY

This invention is a continuation-in-part of U.S. Patent Application No. 61/098,684, filed Sep. 19, 2008, the full disclosure of which are incorporated herein in its entirety.

### BACKGROUND OF THE INVENTION

1. Field of the Invention

The products and methods of the present invention provide a drill for chronic total occlusions (CTO).

2. Description of the Related Art

As the population ages, the prevalence of chronic occlusive disease of the lower extremity increases, significantly influencing lifestyle, morbidity, mortality and health related overall costs. In addition, multiple co-morbid conditions will increase the risks of open surgical procedures.

New endovascular devices have been introduced that are able to cross atherosclerotic obstructions with less morbidity and comparable results with traditional surgery.

However, a close review of the technologies, indications, restrictions and clinical results of all endovascular devices available in the market has shown a variety of disadvantages in each one of them, leaving the necessity to develop new ideas, capable to address all the problems within just one product.

CTO's account for a significant portion of the peripheral vascular atherosclerotic lesions, outnumbering pure stenosis in patients with advanced disease. Patients with this condition may present a wide variety of signs and symptoms, ranging from mild claudication to limb-threatening gangrene. Treatment options typically vary with vessel condition, with the degree varying from dietary and lifestyle changes, to stenting up to surgical bypass. Bypasses are applied to arteries and veins, with bypasses made out of vessels that are grafted to bypasses made out of artificial conduits, including such artificial material as PTFE (polytetrafluoroethylene).

For patients with peripheral artery disease (PAD), the superficial femoral artery (SFA) is one of the most commonly involved arteries. PAD affects at least 8 to 10 million Americans each year.

When evaluating patients with PAD, physicians are often faced with long segments of atherosclerotic occlusions, and treatment success is directly related to the extension, location and pattern of these lesions.

To address this problem, a new classification for PAD was developed by a multidisciplinary group from North America and Europe, forming the Transatlantic inter-Society Consensus (TASC) task force, which published a new classification guidelines for atherosclerotic disease.

Based on these guidelines, femoropopliteal lesions were divided into four types:

Type A lesions are single focal lesions less than 3 cm in length not involving the origins of the SFA or the distal popliteal artery;

Type B lesions are single lesions 3 to 5 cm in length not involving the distal popliteal artery or multiple or calcified lesions less than 3 cm in length;

Type C lesions are single and more than 5 cm in length, or multiple lesions between 3 and 5 cm in length with or without calcification;

Type D lesions are those with complete occlusion of the common femoral artery (CFA), SFA or popliteal artery.

Occlusion of the SFA occurs in many patterns, although three variations are the most common. The first is varying length segmental occlusions at the adductor canal level.

The second variation is seen with occlusion from the origin (CFA) down to the popliteal artery.

The third is varying length occlusions involving both the SFA and popliteal artery.

An occluded SFA frequently contains several areas of severe stenosis or short segment occlusions, with one or more areas of thrombosis, formed by old and relatively recent clots.

Most long SFA occlusions begin with a proximal stump, followed by varying degrees of distal vessel reconstitution in the form of collaterals from the profunda femoral artery (PFA).

Indications for treatment include all patients with symptoms varying from limiting claudication, rest pain, to limbs with mild (focal) to severe (extensive) signs of gangrene.

Until recent years, bypass surgery was the commonest option to treat these patients. Many recent advances in endovascular technology, however, have increased the interventional armamentarium to treat CTO, replacing surgery in a large number of therapeutic procedures, but results are still sub-optimal.

The ability to successfully cross long total occlusions and re-enter the true lumen is directly related to acute procedural success and long term patency and, up to this date, clinical data are not encouraging. Technical success for crossing long SFA occlusions ranges from 15 to 60%, depending on lesion length, calcification, runoff vessel status and operator experience.

Current technologies include various systems for advancing a device through the CTO. These include:

1. Glidewire Technique:

The simplest recanalization strategy remains the combination of a hydrophilic guide wire combined with the support of a low-profile catheter. If successful intraluminal re-entry into the distal reconstitution site is not achieved, a re-entry device is a consideration for completion of the case. For long and calcified obstructions, the sole use of a Glidewire to transverse the occlusion is expected to present lower success rates (as low as 10%).

2. SafeCross Wire:

This wire has the unique property of an optical coherence reflectometer. It is combined with radiofrequency energy that is delivered from the tip if the reflective signal obtained by the sensor identifies a luminal position. The benefit is the advantage of remaining in the intraluminal space, thus reducing the dissection plane of a long occlusive lesion.

3. Excimer Laser:

It has a direct effect on platelets by reducing their aggregation. The benefit of uncovering the true lesion in a long occlusive segment reduces stent implants.

4. Frontrunner:

It is a blunt micro dissection device that takes advantage of the elastic properties of adventitia versus inelastic properties of fibrocalcific plaque to create fracture planes, separating atherosclerotic plaque in various tissue planes, creating a passage through CTO.

5. Pioneer Catheter:

It is a 6.2Fr catheter with two wire ports, one with a hollow core nitinol needle that is guided by an integrated phased-array intravascular ultrasound device (IVUS), enabling vessel imaging, which guides the re-entry site and the needle orientation to true lumen. Difficulty in successful re-entry is encountered with highly calcified vessels, poorly visualized distal reconstitution, deep subintimal catheter location and poor wire angle.

6. Outback Catheter:

It is a 6.0Fr compatible catheter used for re-entry with a hollow 22-gauge canula for distal vessel puncture, using fluoroscopic imaging. It presents the same difficulties as with the Pioneer catheter.

7. SilverHawk:

A debulking technique is plaque excision using this atherectomy catheter, which is a monorail device with a carbide cutting blade at the tip that spins at 8.000 rpm when activated. It is a forward-cutting system and the excised plaque is collected into the nose of the catheter and removed from the body. Expense is an issue. More than one catheter is often required, at a cost of up to $3.000 per catheter. Another issue is the possibility of artery wall damage with high speed atherectomy blades.

8. Polarcath Cryoplasty:

Cryoplasty combines angioplasty and cold therapy using liquid nitrous oxide as the balloon inflation media. The potential advantages include altered plaque response with more uniform vessel dilation and less medial injury, reduced elastic recoil, positive vessel remodeling and smooth muscle cell apoptosis.

It is not considered a primary device, since it needs the space through the plaque to be advanced, solely achieved by means of other recanalization systems and devices. However, it has the advantage to successfully complement other procedures and ensure long-term patency rates to the treated area.

9. Thrombolytic Therapy:

Thrombolytic therapy is still the treatment of choice in acute arterial occlusions, despite the use mechanical thrombectomy devices. There are only a few reports regarding thrombolytic therapy in CTO, but it has changed in the past years, partially due the introduction of new endovascular technologies. It is often difficult to predict the occlusive elements in an occlusion seen on angiograms (calcified stenotic plaques, old thrombosis and new centered clots). Thrombolysis is the key to a successful recanalization, because it enables identification of the variable patterns present in an occlusion.

10. Surgery:

Under certain conditions, surgical revascularization remains a superior treatment option compared to the endovascular intervention devices available, due in part to its proven long-term clinical success and the lack of a safe and effective single recanalization system for endovascular use.

Up to the present date, clinical scenarios in which surgical revascularization should be considered a primary treatment include: multilevel disease with tissue loss or gangrene; CFA atherosclerosis; long-segment SFA occlusion; popliteal artery disease and diffuse tibial vessel occlusive disease.

None of the prior approaches provide a combination of simplicity with the ability to transverse long and calcified occlusive lesions in a cost effective manner while avoiding the pitfalls described.

### SUMMARY OF THE INVENTION

The invention describes a device for treating total occlusions of severe peripheral vessel disease.

The device generally comprises: (a) an external conduit comprising a proximal end, a distal end, a cylindrical outer surface sized for passage through a blood vessel, and an internal lumen, the conduit lumen being threaded within at least a portion of the length of the conduit at the distal end, and (b) a shaft comprising a proximal end, a distal end, an internal lumen, and a cylindrical outer surface fitted inside the conduit lumen and having external threading over at least a portion of the length of the shaft at the distal end; where the shaft threading engages the external conduit threading allowing the shaft to be advanced through the lumen by rotation of the shaft.

In a different embodiment, the invention provides such a device where the treatment is a medical procedure selected from the group of procedures comprising penetration of an organized, total occlusion, penetration of a thrombus, and installation of a guidewire.

In a different embodiment, the invention further provides such a device where the shaft is turned at a rate permitting very low speed rotational treatment of the obstructed vessel.

In a different embodiment, the invention still further provides such a device where the shaft is torsionally reinforced.

In a different embodiment, the invention further provides such a device where the shaft comprises a surgical tool for penetration of the thrombus.

In a different embodiment, the device further comprises: (c) an internal conduit fitted inside the shaft lumen and comprising a proximal and distal end.

In a different embodiment, the invention further provides such a device where the internal conduit comprises a guide wire.

In a different embodiment, the invention further provides such a device where the internal conduit distal end comprises an externally controlled steering tip.

In a different embodiment, the invention further provides such a device where the steering tip is selectably bendable.

In a different embodiment, the invention still further provides such a device where the guide is responsive to external control to effectuate a bend in the guide, thereby changing the orientation of the steering tip.

In a different embodiment, the invention further provides such a device where the guide is responsive to mechanical control.

In a different embodiment, the invention further provides such a device where the guide is responsive to electrical control.

In a different embodiment, the invention further provides such a device where the guide is responsive to pressure control.

In a different embodiment, the invention further provides such a device where the guide is responsive to pressure created within the shaft interior.

In a different embodiment, the device further comprises: (c) an expandable locating element at the distal end of the conduit.

In a different embodiment, the invention further provides such a device where the expandable element is capable of being selectively expanded to engage the walls of the vessel behind a region of occlusion.

In a different embodiment, the invention further provides such a device where the expandable element is produced of polymeric material.

In a different embodiment, the invention further provides such a device where the expandable element comprises a low-pressure balloon.

In a different embodiment, the invention further provides such a device where the low pressure is expandable from pressure applied within the conduit.

In a different embodiment, the invention further provides such a device where the low pressure balloon provides a stable and centered holder and platform for a variety of tools through inflation within the vessel.

In a different embodiment, the invention further provides such a device where the expandable element is a mechanical element.

In a different embodiment, the invention further provides such a device where the blood vessel is partially occluded.

In a different embodiment, the invention further provides such a device where the blood vessel is totally occluded.

In a different embodiment, the invention further provides such a device where the device creates a passage through a vascular occlusion.

In a different embodiment, the invention further provides such a device where one of the small secondary lumen is connected via external ports to the low pressure polymeric balloon at the distal end.

In a different embodiment, the invention further provides such a device where one of the small secondary lumen is connected via an external port to the workspace constrained by the vessel wall, thrombus and inflated low pressure polymeric balloon providing for delivery of solutions or medication to the work site.

In a different embodiment, the invention further provides such a device where the external conduit further comprises a conduit with the distal end of the external conduit manufactured with straight or pre-formed curvature of varying degree of bend to provide for correct axial alignment of the distal end upon inflation of the low-pressure polymeric locating balloon.

In a different embodiment, the invention further provides such a device where the external conduit further provides the ability to withdraw and re-insert tools, change tools and be assured of a repeatable presentation to the thrombus.

In a different embodiment, the invention further provides such a device where the external conduit further provides closure or isolation of the vessel treatment area assuring no entry of material into the bloodstream and an opportunity to introduce medication directly to the treatment area without dilution. The introduced medication would be compartmentalized and held at the site and not dispersed throughout the bloodstream.

In a different embodiment, the external conduit further provides for, through inflation of the external polymeric low pressure balloon at the distal end, the external sheath serving as an anchored stable tool platform in the vessel serving allowing the external sheath to serve as a backstop or thrust block thus allowing generation of axial penetrating force for tool advancement via rotation of various tools within the threaded internal lumen.

In a different embodiment, the invention further provides such a device where the external conduit further provides for precise, reliable and controlled advance of tools through engagement of the internal threads located within the internal lumen at the distal end via low speed rotation of various tools within the threaded lumen.

In a different embodiment, the invention further provides such a device where the shaft is torsionally reinforced.

In a different embodiment, the invention further provides such a device where the distal end of the shaft shall have affixed various tools for penetration of the thrombus.

In a different embodiment of the invention, the torsionally reinforced shaft further comprises a braid reinforced, flexible tube with external threads at the distal end of sufficient length to provide for external thread engagement for the full distance of the expected tool advancement.

In a different embodiment of the invention, the torsionally reinforced shaft further comprise an option for attachment of a variety of mechanical, ultrasonic, laser or other tools at the distal end.

In a different embodiment, the torsionally reinforced shaft further comprises a braid reinforced, flexible tube with internal lumen of sufficient size to accommodate a steering device or standard guidewire.

In a different embodiment, the invention further provides such a device where the torsionally reinforced shaft further provides for axial penetrating force for tool advancement being generated through the external threads engaged both within the external sheath and the thrombus itself as the tool advances.

In a different embodiment, the invention further provides such a device where the torsionally reinforced shaft further provides for penetration of totally occluded, organized fibrotic thrombus via a screw principle with external threads engaged in the external multi-lumen conduit and in the thrombus itself thereby generating greater axial penetrating force in situations where the thrombus is more organized and fibrotic.

In a different embodiment, the invention further provides such a device where the torsionally reinforced shaft further provides for penetration of totally occluded, organized fibrotic thrombus via axial penetrating force generated via the external threads and torsional loading of the reinforced shaft rather than columnar loading.

In a different embodiment, the invention further provides such a device where the torsionally reinforced shaft further provides for penetration of totally occluded, organized fibrotic thrombus via axial tool advancement forces generated through exploitation of the torsional strength and rigidity of the material. This application, coupled with the control provided via the external threads, make it possible to advance the tool without “forcing” it thus allowing exceptional control.

In a different embodiment, the invention further provides such a device where the torsionally reinforced shaft further provides for penetration of totally occluded, organized fibrotic thrombus via a tool tip which need not be particularly sharp as the penetration is being accomplished through compression of the thrombus material rather than incision.

In a different embodiment, the invention further provides such a device where the internal conduit further comprises a reinforced, flexible tube where the bendable steering tip is responsive to external control thereby changing the direction of the torsionally reinforced shaft.

In a different embodiment, the invention further provides such a device where the internal conduit steering device further comprises a reinforced, flexible tube where the tube is described by a small external diameter thus allowing the steering device to occupy a small internal lumen within the torsionally reinforced shaft which carries the advancing tool.

In a different embodiment, the invention further provides such a device where the internal conduit steering device further provides for a unique concept of steering through differential stiffness tubing materials and pressurization.

In a different embodiment, the invention further provides such a device where the internal conduit steering device further provides for better controllability through finite control of the steering pressure.

In a different embodiment, the invention further provides such a device where the internal conduit steering device further provides for simple manufacture via use of variable stiffness tube.

In a different embodiment, the invention further provides such a device where withdrawal of the internal conduit steering device from the torsionally reinforced shaft after tool advancement the steering device allows the internal lumen of the torsionally reinforced shaft internal lumen open for insertion of a guidewire, infusion catheter or other tool.

The invention also provides a method for performing a medical procedure at the site of an occluded blood vessel, the method comprising: (a) advancing a conduit into a patient's vasculature to a location near the occlusion, the conduit comprising a proximal and a distal end, and a guide wire internal to the conduit for performing a medical procedure at the site of the occlusion; and (b) providing an expandable element located at region adjacent to the conduit distal end, where the expandable element can be selectively expanded to engage the walls of the vessel at a region adjacent the occluded region, whereby the device is stabilized within the blood vessel by the engagement of the expandable element and the vessel.

In one embodiment, the invention further provides such a method where the blood vessel is partially occluded.

In a different embodiment, the invention further provides such a method where the blood vessel is totally occluded.

In a further embodiment, the invention further provides such a method where the procedure is angioplasty.

In a different embodiment, the invention further provides such a method where the procedure is ablation of the occlusion.

In a still different embodiment, the invention further provides such a method where the procedure involves cutting of the occlusion.

In a different embodiment, the invention further provides such a method where the procedure involves applying a drug at a region of the occlusion.

In a different embodiment, the invention further provides such a method where the drug is held at the region by blockage of fluid passage by the expandable element.

In a different embodiment, the invention further provides such a method where the surgical element is a device for reducing the occlusion.

In another embodiment, the invention further provides such a method where the surgical element is a device for creating a passage through the occlusion.

In a different embodiment, the invention further provides such a method where the surgical element is affixed to a guide wire.

In a different embodiment, the invention further provides such an improvement where the device has an internal passage and the guide wire is located within the internal lumen.

These and other features and advantages of this invention are described in, or are apparent from, the following detailed description of various exemplary embodiments of the apparatus and methods according to this invention.

### BRIEF DESCRIPTION OF THE DRAWINGS

The attached figures are provided to show the construction and operation of the total occlusion drill described herein.

FIG. 1 shows the main components of the assembled device.

FIG. 3 shows the external conduit component of the device depicted in FIG. 1.

FIG. 3 shows the external conduit of FIG. 3, only with expandable element inflated for engaging a vessel wall.

FIG. 4 shows the threaded shaft of the device in FIG. 1.

FIG. 5 shows the steering catheter of the device in FIG. 1.

FIG. 6 shows the operation of the steering tip of the catheter shown in FIG. 5.

FIG. 7 shows the assembled device of FIG. 1 under actuation to provide steering control.

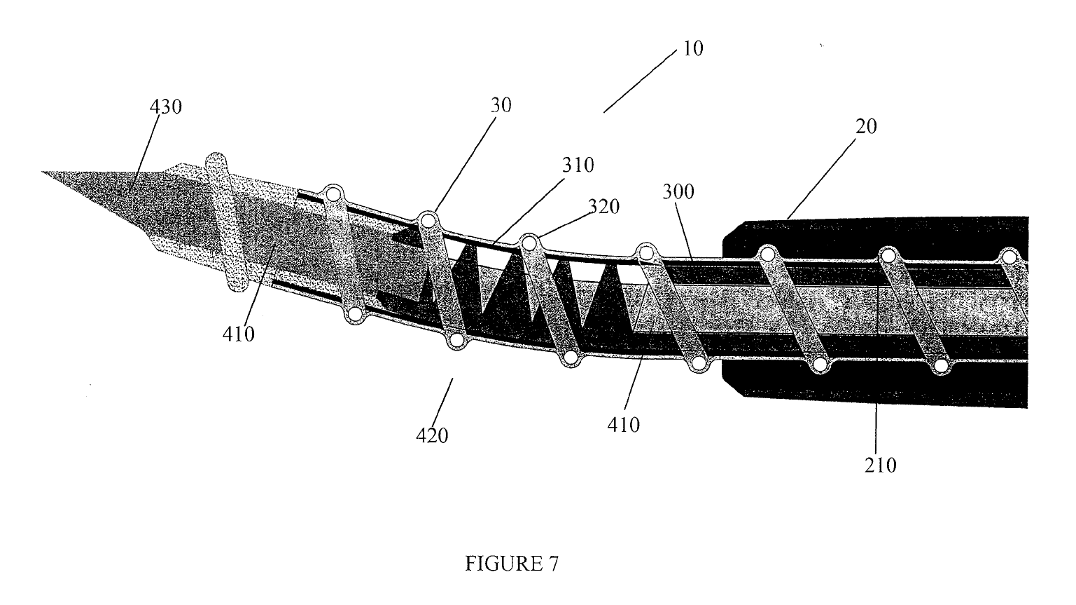

FIG. 8 depicts the positioning of the device adjacent an occlusion and anchored by the expandable component within the vessel wall.

FIG. 9 depicts the further operation of the device shown in FIG. 8, with the shaft advanced into the occlusion.

### DETAILED DESCRIPTION OF THE INVENTION

This invention provides a device for minimally invasive procedures for the treatment of severe peripheral vascular disease, which has become an important problem for an increasing number of patients in our aging population, including all types of organized fibrous occlusions in blood vessels, particularly CTOs.

The device is catheter-based, cost effective and simple to operate, and can effectively treat all types of occluded vessels and various lesions in any location, with minimal morbidity. Having relatively few coordinating parts, the device is simple to manufacture via use of variable stiffness tubings and guidewires.

The device also enables identification of the variable patterns present in an occlusion, uncovering the true lesion in a long occlusive segment, reducing the need for stent implants.

The device is capable of creating a passage through the real chronic total occlusion, while avoiding arterial wall damage, and can transverse long and calcified occlusive lesions, while remaining confined to the intraluminal space, reducing the dissection plane of a long occlusive lesion.

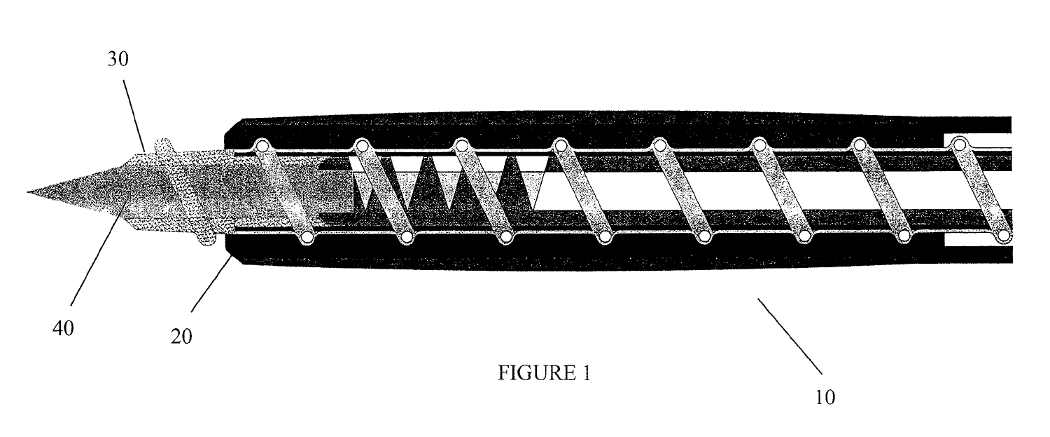

In reference now to the figures, particularly FIG. 1, there is shown a vascular device 10 that provides for rotational treatment and penetration of an organized, total occlusion, thrombus and installation of a guidewire.

The device 10 has an external, multi-lumen conduit, or sheath 20, a threaded shaft or cutting tool 30 and a surgical device or steering catheter 40.

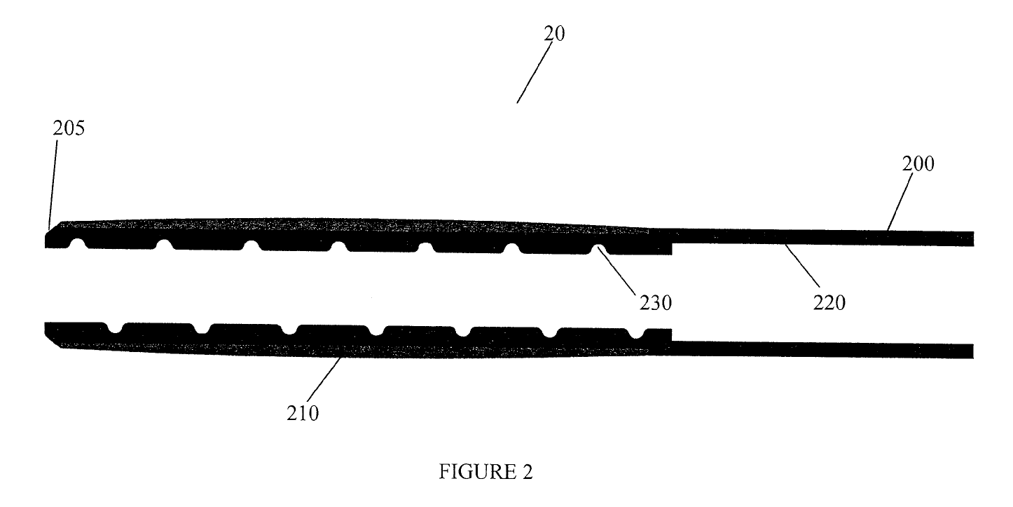

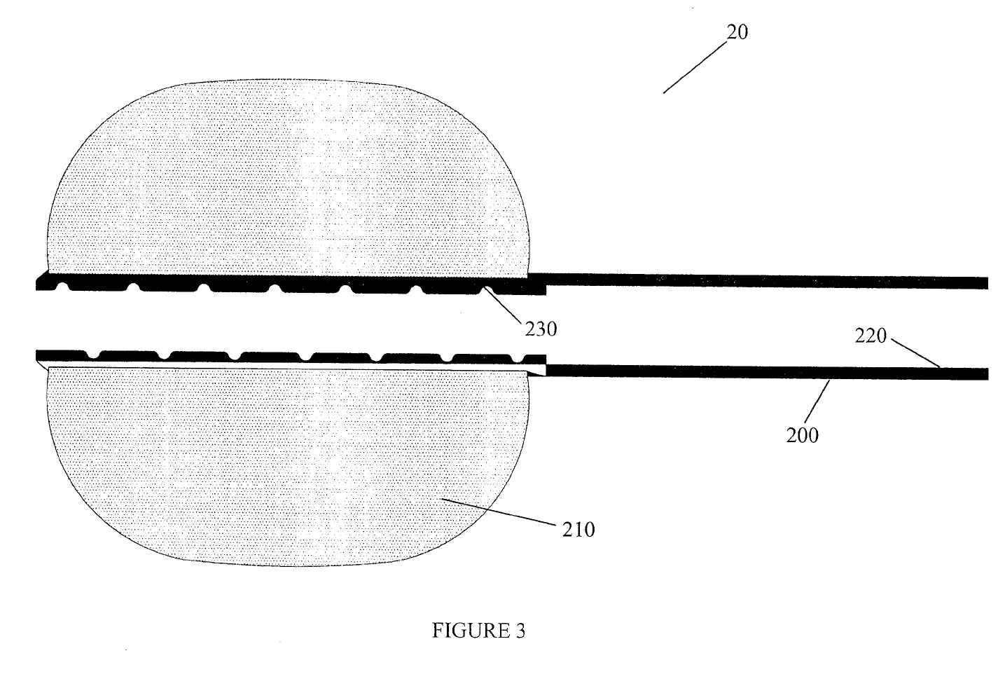

In reference now to FIGS. 2 and 3, the sheath 20 generally has a cylindrical outer surface 200 elongated with distal 205 and proximal ends (not shown). An engagement element, such as an external low-pressure polymeric locating balloon 210 is located at the distal end. The sheath 20 also comprises an internal lumen 220 having internal threads 230 over at least a portion of the length of the internal lumen at the distal end 205.

In particular reference to FIG. 3, the external polymeric low pressure balloon 210 can be inflated, and through inflation within the vessel will provide a stable and centered holder and platform for a the application of a variety of tools through the lumen 220.

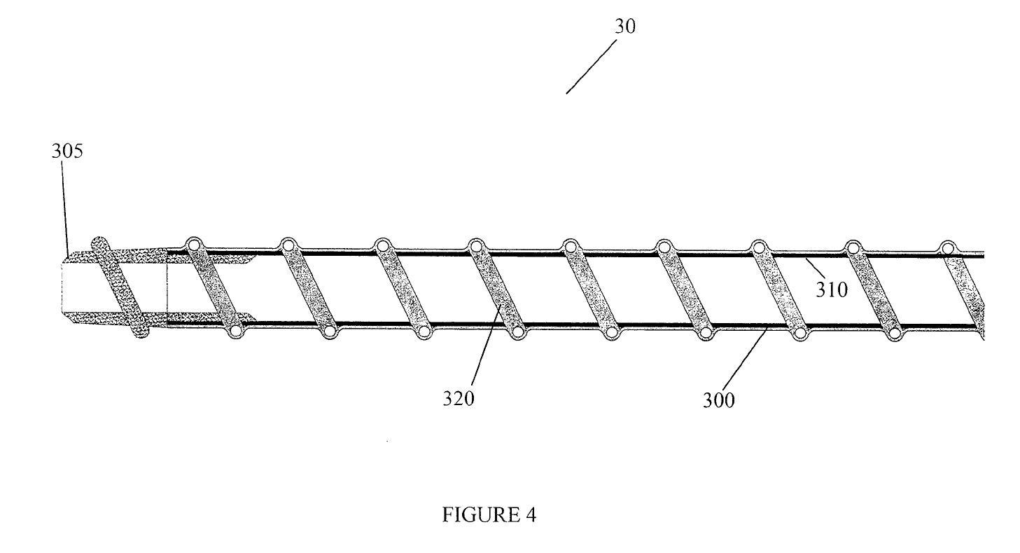

In reference now to FIG. 4, there is seen the torsionally reinforced shaft 30 with a single internal lumen 310 fitted inside the primary lumen 220 of the external conduit 20. The shaft 30 has main body 300 with a proximal end 305, and a distal end (not depicted), with external threading 320 over at least a portion of the length of the shaft 30 at the distal end 305. The threading 320 engages the internal threading 230 of the external conduit 20 allowing the shaft 30 to be advanced via rotation. The distal end 305 of the shaft 30 may also be affixed with various tools for cutting abrasion or otherwise the reduction or penetration of the occlusion.

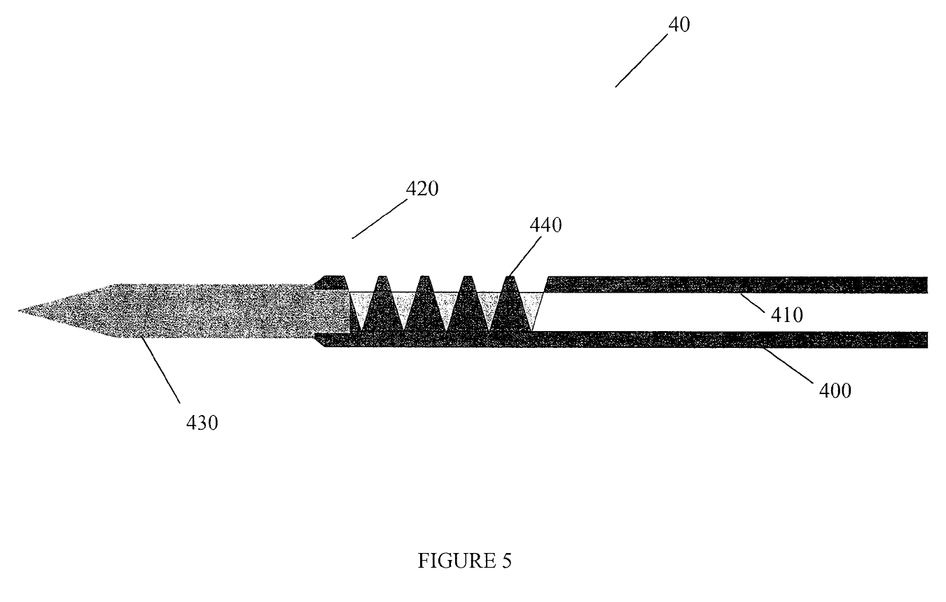

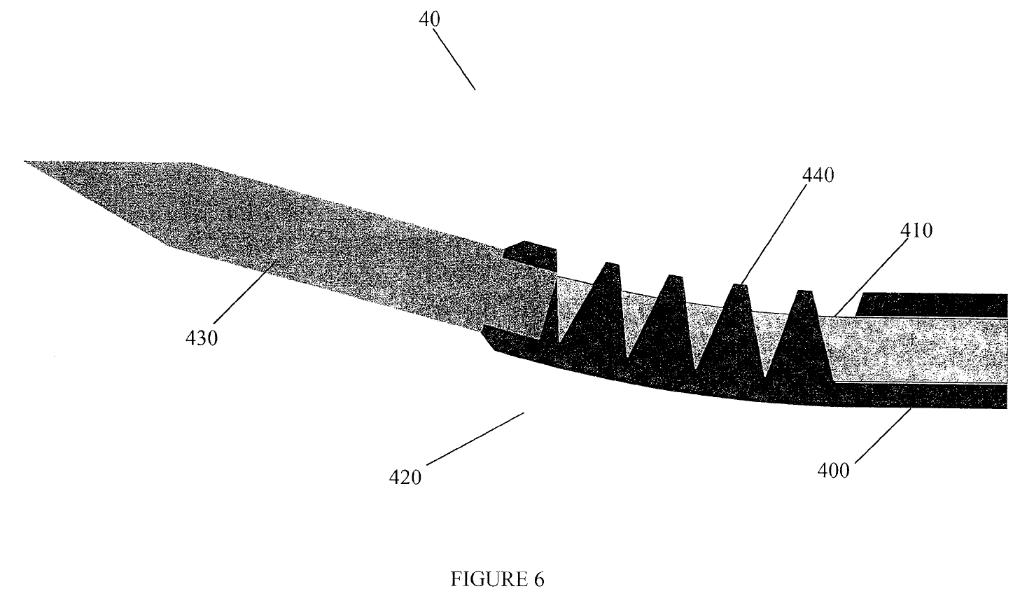

The shaft 30 forms a flexible tube with internal lumen 310 of sufficient size to accommodate a steering device or standard guidewire. Turning now to FIGS. 5 and 6, a steering catheter 40 is depicted. As seen from FIG. 1, this device is located within the internal lumen 310 of the shaft 30. In reference again to FIGS. 5 and 6, it is seen that the steering catheter 40 has an external surface 400 with an internal lumen 410 and a bendable steering tip 420 located near the distal end 430. The proximal end is not depicted. Preferred is a stainless steel cutting tip. The sharpened tip of the distal end 430 helps the tool advance and provides support and steering for the threaded cutting too.

Pneumatic activation through the lumen 410 can be used to control the amount of bending and thus direction of the tip 420 while allowing good flexibility in the remainder of the shaft. For instance, the tip may be provided with a pre-formed curvature 440 that responds to pneumatic pressure in the lumen 410. The pre-formed curvature 440 may be provided with varying degrees of bend to provide for correct axial alignment of the distal end 430. It is important that the distal end 430 be properly oriented upon inflation of the low-pressure polymeric locating balloon 210.

The lumen 220 or internal lumen 310 or 410 of the secondary tools may also be connected via external ports to provide the pressure to inflate the low pressure polymeric balloon 210. The small secondary lumen 310 and 410 may also provided access to the external port and provide for delivery of solutions or medication to the work site.

Once engaged, the external conduit 20 further provides the ability to withdraw and re-insert other tools, while being assured of a repeatable presentation to the thrombus.

The balloon 210 and external conduit 20 further provide closure or isolation of the vessel treatment area assuring no entry of material into the bloodstream and an opportunity to introduce medication directly to the treatment area without dilution. The introduced medication can be compartmentalized and held at the site and not dispersed throughout the bloodstream.

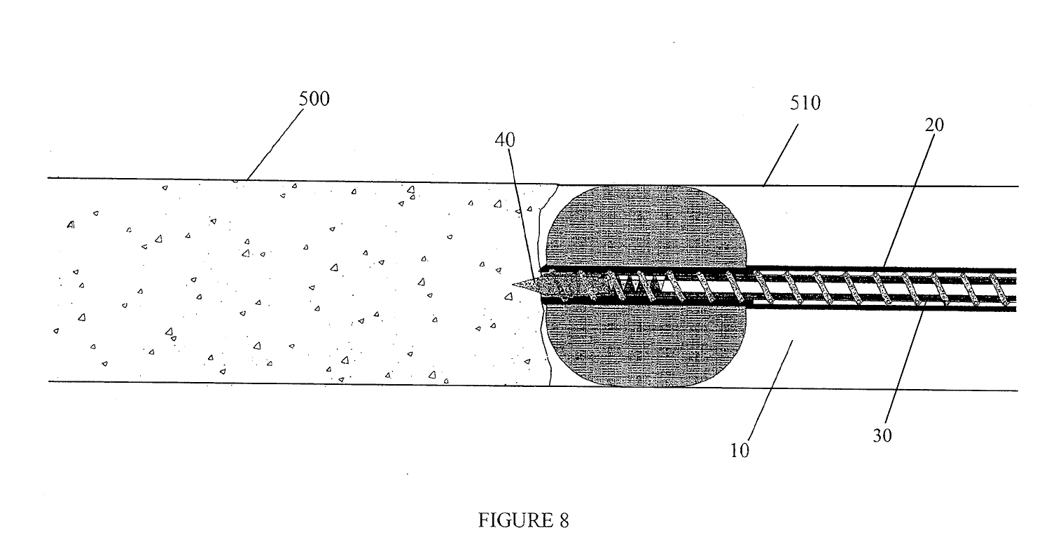

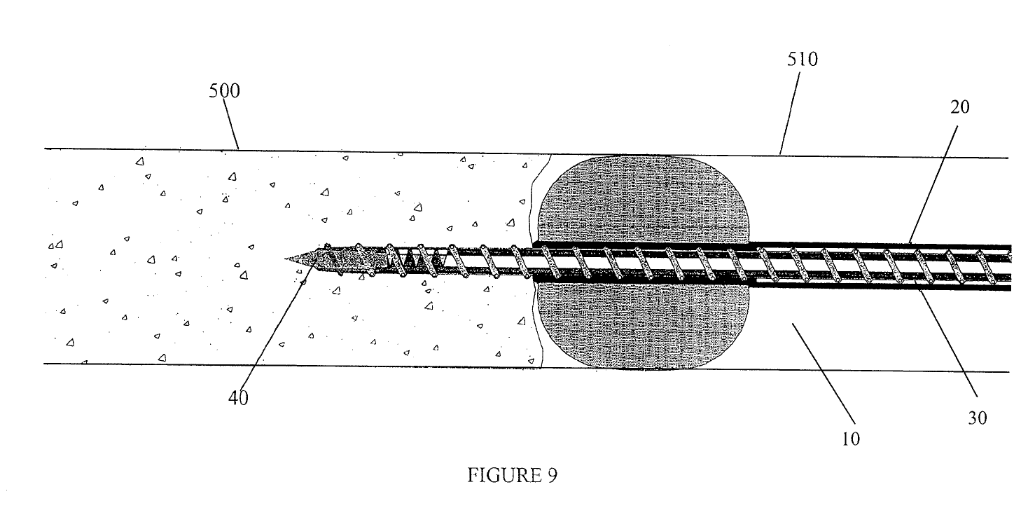

As seen now in reference to FIGS. 8 and 9, through inflation of the external polymeric low pressure balloon 210 at the distal end 205, the external conduit 20 further serves as an anchored stable tool platform at the site of a thrombus or occlusion 500 within in the vessel 510. The balloon 210 and conduit 20 serve as a backstop or thrust block, allowing generation of axial penetrating force for tool advancement via rotation of various tools within the threaded internal lumen, of instance the advance of the shaft 30 by rotation and engagement within the guide 20. The change from FIG. 8 to FIG. 9 depicts the advancement of the cutting shaft 30 after eight full rotations within the conduit 20.

By stabilizing the external conduit 20, the device 10 allows precise, reliable and controlled advance of tools through engagement of the internal threads 230 located within the internal lumen 220 via low speed rotation.

The shaft 30 is preferably a torsionally reinforced shaft, in the form of a braid reinforced, flexible tube with external threads at the distal end of sufficient length to provide for external thread engagement for the full distance of the expected tool advancement. The torsionally reinforced shaft 30 may further comprise an option for attachment of a variety of mechanical, ultrasonic, laser or other tools at the distal end.

As the tool advances, the torsionally reinforced shaft 30 provides for axial penetrating force for tool advancement being generated through the external threads engaged both within the external sheath 20 and the thrombus 500 itself.

The torsionally reinforced shaft 30 further provides for penetration of totally occluded, organized fibrotic thrombus 500 via a screw principle with external threads engaged in the external multi-lumen conduit 20 and in the thrombus 500 itself thereby generating greater axial penetrating force in situations where the thrombus 500 is more organized and fibrotic.

The torsionally reinforced shaft 30 further provides for penetration of totally occluded, organized fibrotic thrombus 500 via axial penetrating force generated via the external threads and torsional loading of the reinforced shaft 30 rather than columnar loading.

The torsionally reinforced shaft 30 also provides for penetration of totally occluded, organized fibrotic thrombus 500 via axial tool advancement forces generated through exploitation of the torsional strength and rigidity of the material. This application, coupled with the control provided via the external threads, make it possible to advance the device 10 without “forcing” it thus allowing exceptional control.

The torsionally reinforced shaft 30 may also provide for penetration of totally occluded, organized fibrotic thrombus 500 via a tool tip 430 which need not be particularly sharp as the penetration is being accomplished through compression of the thrombus material rather than incision.

In this fashion, the device 10 provides controllability through finite control of the steering pressure. The screw movements given by the operator are totally under fluoroscopic control and may be interrupted at any time. The unique characteristic of the device is to be under the operators control at all times of use and without any external energy applied, only that coming from the operator's hand, which gives the device superior safety and effectiveness.

Withdrawal of the internal conduit steering device 40 from the torsionally reinforced shaft 30 after tool advancement the steering device allows the internal lumen 310 of the shaft 30 open for insertion of a guidewire, infusion catheter or other tool

A standard drill system of the invention may contain other elements, as are conventionally known. For instance, a long guiding sheath (straight or crossover, not depicted), may be used to gain easy access to the occluded area and protect minor stenotic areas of dissection during the procedure due to catheter exchange. In that event, the sheath is pushed forward during the procedure to keep the previous described characteristics, making it necessary to have a longer device.

Similarly, a long and standard hydrophilic guidewire with curved tip can be used as a primary procedure before and after the use of the device.

The well established superiority of the hydrophilic wires to cross stenotic or more difficult areas make it the device of choice to facilitate the entire procedure. It must be a stiff wire in order to give better support and torque during the sheath and device 10 advancing.

A regular low-profile hydrophilic guiding catheter, which comes with a gold marker at the tip to provide good visualization of its position during the procedure.

Where desirable, the steering tip 430 may be replaced by a drilling head with different designs (spherical, oval, conical, etc.) mounted at the tip of a straight stiff hydrophilic wire, giving it the ability to “drill” into the thrombus 500 with low, manually controlled rotational movements that gives its major characteristics of opening a way through the occluded areas without major risks of vessel wall damage.

Finally, catheter directed thrombolysis and heparin can be adjunctive in the entire process.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

1. A medical device for treatment of an obstructed vessel, said apparatus comprising:
(a) an external conduit comprising a proximal end, a distal end, a cylindrical outer surface sized for passage through a blood vessel, and an internal lumen, said conduit lumen being threaded within at least a portion of the length of said conduit at said distal end, and
(b) a shaft comprising a proximal end, a distal end, an internal lumen, and a cylindrical outer surface fitted inside the conduit lumen and having external threading over at least a portion of the length of said shaft at said distal end;
wherein said shaft threading engages said external conduit threading allowing said shaft to be advanced through said lumen by rotation of said shaft.

2. The device of claim 1, wherein said treatment is a medical procedure selected from the group of procedures comprising penetration of an organized, total occlusion, penetration of a thrombus, and installation of a guidewire.

3. The device of claim 1, wherein said shaft is turned at a rate permitting very low speed rotational treatment of said obstructed vessel.

4. The device of claim 1, wherein said shaft is torsionally reinforced.

5. The device of claim 1, wherein said shaft comprises a surgical tool for penetration of the thrombus.

6. The device of claim 1, further comprising:
(c) an internal conduit fitted inside the shaft lumen and comprising a proximal and distal end.

7. The device of claim 2, wherein said internal conduit further comprises a guide wire.

8. The device of claim 4, wherein said internal conduit distal end comprises an externally controlled steering tip.

9. The device of claim 4, wherein said steering tip is selectably bendable.

10. The device of claim 5, wherein said guide is responsive to external control to effectuate a bend in said guide, thereby changing the orientation of said steering tip.

11. The device of claim 6, wherein said guide is responsive to mechanical control.

12. The device of claim 6, wherein said guide is responsive to electrical control.

13. The device of claim 6, wherein said guide is responsive to pressure control.

14. The device of claim 9, wherein said guide is responsive to pressure created within the shaft interior.

15. The device of claim 1, further comprising:
(c) an expandable locating element at said distal end of said conduit.

16. The device of claim 11, wherein said expandable element is capable of being selectively expanded to engage the walls of said vessel behind a region of occlusion.

17. The device of claim 12, wherein said expandable element is produced of polymeric material.

18. The device of claim 12, wherein said expandable element comprises a low-pressure balloon.

19-47. (canceled)

48. In a medical device for treating an occluded blood vessel, said device comprising a distal and proximal end, said distal end having an element used in performing a procedure on an occluded region of a blood vessel, the improvement comprising providing an expandable element located at region adjacent to said conduit distal end, wherein said expandable element can be selectively expanded to engage the walls of said vessel at a region adjacent said occluded region, whereby said device is stabilized within said blood vessel by the engagement of said expandable element and said vessel.

49. The improvement of claim 48, wherein said expandable element is produced of elastic material secured over said conduit external wall.

50-100. (canceled)
